(19) United States
(12) Patent Application Publication
(10) Pub. No.: US 2005/0090754 A1
(43) Pub. Date: Apr. 28, 2005

(54) Body worn latchable wireless medical computing platform

(76) Inventors: Steven B. Wolff, Pt. Roberts, WA (US); J. James Guzzetta, El Sobrante, CA (US)

Correspondence Address: J. JAMES GUZZETTA, 4176 SANTA RITA ROAD, EL SOBRANTE, CA, 94803, US

(21) Appl. No.: 10/937,535

(22) Filed: Sep. 8, 2004

Related U.S. Application Data
(60) Provisional application No. 60/501,621, filed on Sep. 8, 2003.

(51) Int. Cl. A61B 5/04

(52) U.S. Cl. 600/509

(57) ABSTRACT

A non-invasive body worn computing platform is capable of long term unattended operation to capture, analyze, store, biosensor data and communicate health and system events, ECG waves and other biomedical data from the patient recorded, produced, or analyzed. The non-invasive body worn computing platform operates as a node within a wireless distributed collaborative network. The body worn computing platform has a latch-able mechanical and electrical interface for rapid swapping of the device and power source to and from biosensors, body harness, and power sources.

### REFERENCES CITED

U.S PATENT DOCUMENTS

| | | | | | |
|---|---|---|---|---|---|
| 6,605,038 | . . . health well . . . | Aug. 12, 2003 | Teller | 600/300 | BodyMedia |
| 6,694,180 | . . . biopotential . . . | Feb. 17, 2004 | Boesen | 600/547 | Boesen |
| 6,225,901 | “Reprogrammable | May 1, 2001 | Kail, IV | 340/539.11 | Cardionet |
| 20030122677 | “Reprogrammable | Jul. 31, 2003 | Kail, IV | 340/573.1 | Cardionet |
| 20040073127 | “Wireless ECG . . . | Apr. 15, 2004 | Istvan e | 600/513 | GMP |
| 5,687,734 | . . . patient | Nov. 18, 1997 | Dempsey | 600/509 | HP |
| | monitor . . . | | | | |
| 6,567,680 | . . . elec. posit. . . . | May 20, 2003 | Swetlik | 600/382 | Medical Da. |
| 8,217,525 | Redu. lead . . . | Apr. 17, 2001 | Medema | 600/508 | Medtronics |
| 6,747,561 | “Bodily worn . . . | Jul. 8, 2004 | Reeves | 340/573.1 | Med-Datan. |
| 5,919,141 | “Vital sign . . . | Jul. 6, 1999 | Money | 600/513 | Money |
| 6,611,705 | “Wireless electro. | Aug. 26, 2003 | Hopman | 600/509 | Motorola |
| 6,577,893 | “Wireless med.dia | Jun. 10, 2003 | Besson | 600/509 | Motorola |
| 20030199777 | “Wireless electro. | Oct. 23, 2003 | Hopman | 600/509 | Motorola |
| 6,494,829 | “Physiological | Dec. 17, 2002 | New, Jr. | 600/300 | Nexan |
| | sensor . . . | | | | |
| 6,603,995 | “Body monitoring . . . | Aug. 5, 2003 | Carter | 600/509 | Reynolds |
| 6471,087 | “Remote patient . . . | Oct. 29, 2002 | Shusterman | 221/2 | Shusterman |
| 6,735,464 | “Electrocardiograh | May 11, 2004 | Onoda | 600/509 | Terumo |
| 6,551,252 | “Systems and | Apr. 22, 2003 | Sackner | 600/536 | VivoMetrics |
| | methods . . . | | | | |

### CROSS REFERENCE TO RELATED APPLICATIONS

We claim benefit from the U.S. Provisional Application No. Application No. 60/501,621 filing date Sep. 8, 2003.

### STATEMENT REGARDING FEDERALLY SPONSORED RESEARCH OR DEVELOPMENT

N/A

### REFERENCE TO SEQUENCE LISTING, A TABLE, OR A COMPUTER PROGRAM LISTING APPENDIX

N/A

### FIELD OF THE INVENTION

The present invention relates to non-invasive body worn computing platforms performing medical utility for disease diagnostic and therapy, medical research and sports medicine.

Specifically the invention relates to the capture of body worn biosensor data over extended periods of time, and the processing, analysis and storage of the data, and wireless transmission of health and system events and data to various servers and personnel such as physicians.

The non-invasive body worn computing platforms operates as a node within a wireless distributed collaborative network.

In addition it relates to the quick release and replacement of a body worn computing platform device.

### BACKGROUND OF THE INVENTION

Physicians and bio-medical engineers have devised a large range of systems to measure medical variables in ambulatory patients. Some of the bio-parameters measured are ECG, EEG, breathing, pulse oxyimetry, blood pressure, sound, and motion sensors.

Cardiac analysis of ECG biosensor data is usually done in the hospital or clinic where the patient is resting. Additional analysis is performed where the patient in on a treadmill.

These diagnostic procedures preclude the ability to obtain the ECG data when the patient is ambulatory. Several systems do provide improvements for the ambulatory patient. Data and event recorders such as Holter, Cardionet or King of Hearts systems provide for this.

In addition, these systems typically use a belt or shoulder/chest strap to hold the device and often use an octopus of “loose” cables to connect the device to the set of ECG electrodes.

Several cardiac diseases require longer term monitoring than is currently provided. In addition, Arrhythmia and Ischemia diagnosis ideally requires longer term monitoring while the patient is in an ambulatory mode. Optimum Ischemia diagnosis requires special QRST algorithms as well.

Currently cardiac devices fall into the following categories:
Implantable (Medtronic, Biotronic etc.)
Stationary (Phillips, as used in hospitals, clinics etc)
EMS (as used by the an ambulance crew)
Carry-able (such as Cardionet devices)
StickOn (Boesson, Motorola but not in use)
Latch-able (as relates to the invention)

Several systems provide some improvement for the ambulatory patient. Data, event, and cell based wireless recorders such as Holter, King of Hearts, or Cardionet systems respectively provide for this.

In order for ambulatory patients to wear non-invasive biosensor devices for more than 24 hours the computing platform must be small in dimension, it must be easy for less physically capable patients to retrieve it from a charging station and place it on their body worn ECG harness for example, and finally it must manage a wireless link for low power consumption, RF radiated power, and error recovery.

All of these systems use a standard 3, or more, electrode lead system where the leads are long and loose and the recording device is worn on the belt.

In addition the devices nor electrodes are waterproof nor comfortable for sleeping.

These configurations often do not provide a comfortable experience even when only wearing them for 24 hours.

Each system has disadvantages.

One is that the devices are large, not waterproof and therefore do support long term high patient compliance.

A second disadvantage is that they do not provide on body analysis and alert production and communication.

In addition another disadvantage is that they operate as slave devices rather than as nodes in a networked collaborative computing environment.

Another disadvantage is that they do not provide for filed upgradeable software and application specific software.

Yet another disadvantage is they do not have a small non-intrusive form factor and easy to latch device.

A final disadvantage is there do not use a long term power source or easily swappable batteries.

There are however several efforts to answer some of these disadvantages. Boesson illustrates a small form factor wireless sensor device.

Cardionet also has a patent in the area of a reprogrammable remote sensor monitoring system.

There are several patents covering a wireless ECG device for replacing monitor cables in hospitals from Motorola and GMP.

One is Swetlik, et al. of Medical Data Electronics whose patent covers an integrated and disposable device and harness.

Several groups have patented garment based harnesses which include a device. One is New, Jr., et al. of Nexan Limited another is Sackner, et al. of VivoMetrics, Inc. Each provides a vest type garment with sensors.

Finally one has a device case that is waterproof. Carter of Reynolds Medical Limited provides a O-ring seal so that a sensor cable and a device can be coupled a water proof manner.

To date, no system or device provides all the functions required to obtain a system for the capture, processing, analytics, and communication of biosensor data, comprising; a body worn computing device with an interface to biosensors processing of biosensor signals with real-time analyzer of biosensor signals that operates as a node in a distributed collaborative processing network that is wireless and/or wired communicating to other processors in the network. In addition has network update-able software.

Further a water and body fluid resistant casing is used provided in a non-protruding small form factor.

Uses a rapid and zero insertion force connector and its mechanical interface to a biosensor harness.

Finally uses a long-term operation power source.

Prior art that are relevant to this invention are discussed below in alphabetical order of their companies (assignees).

Teller, et al. of BodyMedia, Inc. in U.S. Pat. No. 6,605,038 “System for monitoring health, wellness and fitness” illustrates the mounting of sensor device on the upper arm.

Boesen in U.S. Pat. No. 6,694,180 “Wireless biopotential sensing device and method with capability of short-range radio frequency transmission and reception” teaches about a device that posses or interface to ECG as narrow uni-potential electrodes as well as external auditory canal temperature sensor, an ear pulse oximeter, and a hypnotic state sensing EEG.

Kail, IV of Cardionet in U.S. Pat. No. 6,225,901 “Reprogrammable remote sensor monitoring system” illustrates a portable monitoring unit capable of communicating with a central monitoring device, and their portable monitoring unit. They teach about a set of activating parameters for an activation condition selected from the group consisting of a pre-selected state for at least one automatic sensor and a request signal from an external source.

Kail, IV of Cardionet in 20030122677 “Reprogrammable remote sensor monitoring system” is an update which adds one-way voice communication.

Istvan et al of GMP in 20040073127 “Wireless ECG system” is a in-hospital use arm mounted device. Very similar to Hopman's, et al. of Motorola in U.S. Pat. No. 6,611,705 “Wireless electrocardiograph system and method”

Dempsey, et al. of HP in U.S. Pat. No. 5,687,734 “Flexible patient monitoring system featuring a multiport transmitter” illustrates a flexible patient monitoring system.

Swetlik, et al. of Medical Data Electronics in U.S. Pat. No. 6,567,680 “Disposable electrocardiogram transmitter device and electrode node placement facilitator” provides for a waterproof and disposable device affixed to a harness of ECG electrodes.

Medema, et al. of Medtronic Physio-Control Manufacturing Corp. in U.S. Pat. No. 6,217,525 “Reduced lead set device and method for detecting acute cardiac ischemic conditions” uses a classifier to evaluate data coming from a reduced ECG sensor lead set.

Reeves of Med-Datanet, LLC in U.S. Pat. No. 6,747,561 “Bodily worn device for digital storage and retrieval of medical records and personal identification” teaches about a jewelry type device storing a users medical records.

Money, et al. in U.S. Pat. No. 5,919,141 “Vital sign remote monitoring device” illustrates a device for remote monitoring of hospitalized patient vital signs such as ECG data, heart rate, pulse, pulse oximetry, temperature, respiration, and blood pressure.

Hopman, et al. of Motorola in U.S. Pat. No. 6,611,705 “Wireless electrocardiograph system and method” teaches about arm-mounted device that connects to a ECG harness. The device transmits to a standard ECG monitor.

Besson et al of Motorola in U.S. Pat. No. 6,577,893 “Wireless medical diagnosis and monitoring equipment” teaches about a device with integrated sensors. A sensor harness is optional.

Hopman, Nicholas C.; et al. of Motorola in 20030199777 “Wireless electrocardiograph system and method” is an update of U.S. Pat. No. 6,611,705.

New, Jr., et al. of Nexan Limited in U.S. Pat. No. 6,494,829 “Physiological sensor array” illustrates another combination of sensor harness and device.

Carter of Reynolds Medical Limited in U.S. Pat. No. 6,603,995 “Body monitoring apparatus” teaches about a body worn device whose case is waterproof.

Shusterman in U.S. Pat. No. 6,471,087 “Remote patient monitoring system with garment and automated medication dispenser” is another garment based monitor device and sensor system. It teaches however about integrated pill dispensing.

Onoda, et al. of Terumo Kabushiki Kaisha in U.S. Pat. No. 6,735,464 “Electrocardiograph system and its communication device” illustrates another device attached to an ECG harness that communicates its data to a workstation.

Sackner, et al. of VivoMetrics, Inc. in U.S. Pat. No. 6,551,252 “Systems and methods for ambulatory monitoring of physiological signs” teaches about an apparel capable of breathing and ECG sensors and contains a computing device.

### SUMMARY OF THE INVENTION

Accordingly, the present invention provides a non-invasive body worn computing platform for medical utility.

The present invention provides for the capture and processing of a wide variety of bio sensor signals, the analysis and categorization of biosensor signals.

In addition the invention determines, health, system and communication events, and processes biosensor signals and tags them for storage and retrieval.

Further this invention uses a small ergonomic, high patient compliance form factor.

In addition the invention can be quickly attached and replaced and it and its connector are water and body fluid resistant.

In accordance with a further aspect of the present invention, it manages communications bi-directionally with other devices, servers, and workstations receives and installs application and system updates.

Further it uses wireless or wired communications links.

The novel elements believed to be characteristic of the present invention are set forth in the claims.

### BRIEF DESCRIPTION OF THE SEVERAL VIEWS OF THE DRAWINGS

Drawings

FIG. 1 Device Illustrates the data flow of the non-invasive body worn computing platform operating as a node in collaborative wireless network.

FIG. 2 Biosensors Illustrates the various biosensors schemes

FIG. 3 Casing, Latch, and Connector Illustrates the mechincall and electrical latches and connectors

FIG. 4 Harness Illustrates the device harness also supporting the biosensors

### REFERENCE NUMERALS IN DRAWINGS

FIG. 1 Device
a non-invasive body worn computing platform (1)
capture (2)
processing (3)
analytics (4)
communication (5)
wide variety of biosensors (6)
biosensor signals (7)
real-time analyzer (8)
real-time analyzer processing biosensor signals (9)
health events (10)
system events (11)
communication events (12)
storage tags (13)
search tags (14)
retrieval tags (15)
real time processing on board the device (16)
bi-directional communications management (17)
communication with other devices (18)
communication with servers (19)
communication with workstations (20)
receipt and installation of a software module (21)
system updates (22)
network of computing nodes (23)
wireless communications (24)
wired communications (25)
computing and communications node (26)
distributed collaborative processing network (27)
network update-able software (28)
CPU performs processing (42)
CPU system includes memory, data I/O, and networking support (43)
real-time OS (44)
networking and collaboration scheduler (45).
node in the collaborative network (46).
primary health event operation from real-time analysis of biosensor signals (47)
biosensor signal analytic process (48)
capturing, analyzing, and producing health events (49) and related data (50)
particular biosensor analytic process (51)
particular biosensor analytic process is installed and configured (52)
loaded from the collaborative network (53)
biosensor data may transferred to other nodes (54) for primary or secondary biosensor signal processing and analytics
bio-parameters measured included ECG, EEG, breathing, pulse oxyimetry, blood pressure, sound, and motion sensors (55)
harness and device configured for related a biosensor (56)
payload data (57)
network communication of wireless or wired communications links (58)
local wireless (59)
long-range wireless (60)
management of wireless links for minimization of power consumption (61)
management of wireless links for RF radiated power (62),
perform data link error recovery (63).
update-able software over the network (64)
update-able software (65)

FIG. 2 Biosensors
plurality and varied biosensors (37)
biosensors integrated into a body worn harness (38)
biosensors integrated into the casing of the device (39)
capture of the biosensor signals via a data acquisition sub-system (40)
signal rate, resolution and signal filtering configured (41) for the type of biosensor selected
biosensors integrated into the device (71)
biosensor positioned for direct access to the patients body (72)

FIG. 3 Casing, Latch, and Connectors
small ergonomic form factor (29)
quick attachment (30)
water and body fluid resistant connector (31)
water and body fluid resistant casing (32)
non-protruding and small form factor (33)
zero insertion force connector (34)
mechanical interface to a biosensor harness (35)
long-term operation power source (36).
device affixed with a quick attaching latch mechanism (73)
zero insertion force latch (74)
water and bodily fluid resistant (75)
biosensors affixed with a quick attaching latch mechanism (76)
water and bodily fluid resistant zero insertion force latch mechanism (77)
rechargeable battery affixed with a quick attaching latch mechanism (78)
device or battery affixed with a quick attaching latch mechanism on to the recharging station (79)
body movement power generator integrated into the structure of the biosensor harness (80).
patient movement power generator voltage is converted (81) into device power patient swappable battery (66)
AC powered recharging station (67)
AC powered direct connected source (68)

FIG. 4 Harness
body worn harness supports biosensors, device, and integrated body movement power generator (82).
device connects mechanically and electrically to the harness through zero insertion force and water and bodily fluid resistant means (83).
body movement power generator (69).
body movement power generator integrated into the biosensor harness (70)

### DETAILED DESCRIPTION OF THE INVENTION

The present invention provides a non-invasive body worn computing platform (1) for medical utility.

The present invention provides for the capture (2), processing (3), analytics (4), and communication (5) of a wide variety of biosensor (6) signals (7).

In addition the invention processes with a real-time analyzer (8) biosensor signals (9) and produces, health (10), system (11) and communication events (12), and then tags them for storage (13), search (14) and retrieval (15).

This invention performs the said processing on board the body worn device and in real time (16).

In accordance with a further aspect of the present invention, it manages communications bi-directionally (17) with other devices (18), servers (19), and workstations (20) receives and installs application (21) and system updates (22) within the network of other computing nodes (23).

Further it uses wireless (24) or wired communications (25) links.

Importantly the invention operates as a node (26) in a distributed collaborative processing network (27).

The aforementioned network of nodes include this invention which operate as a node where the processing is performed on-board the body worn device

In addition has software that is update-able (28) over the network.

Further this invention utilizes a small ergonomic (29), high patient compliance form factor.

In addition the invention can be quickly attached (30) and replaced and it and its connector (31) are water and body fluid resistant.

Further the invention utilizes water and body fluid resistant casing (32) is used and further is provided in a non-protruding (33) small form factor.

In addition the invention uses a rapid and zero insertion force connector (34) for its mechanical interface to a biosensor harness (35).

Finally the invention uses a long-term operation power source (36).

Device

The present invention provides a non-invasive body worn computing platform for medical utility. The device is non-invasive in that it is a device that is affixed by a variety of means, to the surface of the patient's body and is therefore body worn.

The present invention provides for the capture, processing, analytics, and communication of a wide variety of biosensor signals.

The device is electrically and mechanically connected to a plurality of biosensors. Varied biosensors (37) may be configured for use. In addition the biosensors may be integrated (38) into a body worn harness or may be integrated to the casing of the device (39) or in the preferred embodiment both are supported.

The device provides for capture of the signals from the biosensors via a data acquisition sub-system (40). The analog front end, D/A component, and the digital back end (41) comprised of signal rate, resolution and signal filtering are configured for the type of biosensor selected.

A CPU (42) within the body worn device performs the processing. The CPU system (43) includes memory, data I/O, and networking support.

System software includes a real-time OS (44) with networking and collaborative scheduler (45). The collaborative scheduler distributes data and applications for system and medical applications running on a variety of other nodes on the network.

The device in this invention is a node in the said collaborative network (46).

In accordance with a further aspect of the present invention, the device manages communications bi-directionally with other devices, servers, and workstations receives and installs application and system updates within the network of other computing nodes.

Said processing includes system and health operations.

A primary health operation is that of a real-time analyzer processing biosensor signals (47). Each type of biosensor signal has its own analytic process (48) for capturing, analyzing, and producing health events (49) and related data (50). The analytic process pertaining to a particular biosensor (51) is loaded and run when the biosensor is installed and configured (52). The loading of a particular analytic process can be done across the collaborative network (53). Biosensor data may also be transferred to other nodes (54) for primary or secondary biosensor signal processing and analytics.

Some of the bio-parameters measured are ECG, EEG, breathing, pulse oxyimetry, blood pressure, sound, and motion sensors (55). For a bio-parameter configured a biosensor, related harness and device (56) is installed and so is its related biosensor analyzer software load and run within the related device.

The output of the analyzer includes system and importantly health events. Events are communicated to other nodes within the collaborative network for further processing. Events can include payload data such as from biosensor signals (57).

Network communication (58) between members of the collaborative network may use wireless or wired communications links. A preferred embodiment uses a local wireless (59) and long-range wireless (60) links depending on the device. Body worn devices preferentially use local low power.

Another aspect of the invention is that the system must manage any wireless links for minimization of power consumption (61) as well as RF radiated power (62), and perform data link error recovery (63).

Software is update-able over the network (64). Update-able software includes OS, biosensor, analyzer, and application level components (65).

A patient swappable battery (66) supplies power to the device, as well as an AC powered recharging station (67), a AC powered direct connected source (68), and a body movement power generator (69). The body movement power generator is integrated into the biosensor harness (70). In combination the power source is capable of at least 48 hrs to 7 days of operation without intervention.

Biosensors

Some bio-parameters measured are ECG, EEG, breathing, pulse oxyimetry, blood pressure, sound, and motion sensors. For each bio-parameter configured a related biosensor analyzer software is loaded and run within the related device. In addition a physical electrical and mechanical interface for a particular are also configured.

Certain biosensors may be integrated into the device (71). The biosensor integrated into the device is positioned so that for example an ECG biosensor has direct access to the patients body (72). Other biosensors that could utilize this embodiment are blood pressure, and pulse oxyimetry.

Casing, Latch, and Connectors

In addition the invention can be quickly attached and replaced and it and its connectors are water and body fluid resistant.

This invention utilizes a small ergonomic, high patient compliance form factor.

Further it utilizes a water and body fluid resistant casing in a non-protruding small form factor.

The device of this invention are mechanically affixed to the harness with a quick attaching latch mechanism (73) means. The quick attaching latch mechanism utilizes zero insertion force (74) and water and bodily fluid resistant means (75).

The biosensors of this invention is mechanically affixed to the harness of biosensors with a quick attaching latch mechanism (76) means. The quick attaching latch mechanism utilizes zero insertion force, water and bodily fluid resistant, and low electrical resistance means (77).

The power source such as a rechargeable battery of this invention is mechanically affixed to the harness with a quick attaching latch mechanism means (78). The quick attaching latch mechanism utilizes zero insertion force and water and bodily fluid resistant means.

The recharging station of this invention is mechanically affixed to the harness with a quick attaching latch mechanism means (79). The quick attaching latch mechanism utilizes zero insertion force and water and bodily fluid resistant means.

The body movement power generator is integrated with the structure of the biosensor harness (80). Its electrical signal is cabled through the biosensor harness to device connector. The device has means for converting the patient movement power generator voltage supply (81) into device power supply.

Harness

The invention's device in it's preferred embodiment is affixed to a body worn harness that supports biosensors and integrated body movement power generator (82).

The device connects mechanically and electrically to the harness through zero insertion force and water and bodily fluid resistant means (83).

1. A system for the capture, processing, analytics, and communication of biosensor data, comprising;
a body worn computing device;
interface to said biosensors;
processing of said biosensor signals;
real-time analyzer of biosensor signals;
a node in a distributed collaborative processing network;
wireless and/or wired communication to other processors in the network;
update-able software;
water and body fluid resistant casing;
non-protruding small form factor casing;
rapid and zero insertion force connector;
mechanical interface to a biosensor harness; and
long term operation power source.

2. The system of claim 1, wherein the means for a body worn computing device include:
means for affixing said device to a chest worn harness;
means for affixing said device on an arm;
means for affixing said device on a leg; and
means for affixing said device on a head.

3. The system of claim 1, wherein the means for a body worn computing device performs:
bio-signal capture, analytics, storage, and communication

4. The system of claim 2, and including a lead transformation process, but not limited to:
a 5 lead to 12 standard lead;
a EASI to 12 standard lead;
alternative disease optimum lead arrays to 12 standard lead.

5. The system of claim 2, where the real-time analysis includes, but is not limited to, an Arrhythmia event classifier.

6. The system of claim 2, where the real-time analysis includes, but is not limited to, an Ischemia event classifier.

7. The system of claim 2, where the real-time analysis includes, but is not limited to, a Myocardial Infarction event classifier.

8. The system of claim 6, where the real-time analysis includes, but is not limited to, a Acute Cardiac Infarction Time Insensitive Predictive Instrument (ACI-TIPI), a continuous event classifier.

9. The system of claim 1, wherein the means for a body worn computing device include:
health and system event reporting;
local patient and remote physician alerting.

10. The system of claim 1, wherein the means for a body worn computing device includes, but is not limited to:
local data storage;
data forwarded and stored on handhelds;
data forwarded and stored on servers.

11. The system of claim 1, wherein the means for interface to biosensors includes, but not limited to:
biosensors such as ECG, EEG, breathing, pulse oxyimetry, blood pressure, sound, and motion sensors.

12. The system of claim 1, wherein the means for processing of said biosensor signals and other system issues includes, but are not limited to:
health and disease analytics;
system analytics;
bio-signal capture;
data storage;
alert generation;
user interface; and
communications.

13. The system of claim 1, wherein the means for processing of said real-time analyzer of biosensor signals includes, but not limited to:
Ischemia event classifier;
Arrhythmia event classifier;
Myocardial Infarction event classifier;
a continuous event classifier such as Acute Cardiac Infarction Time Insensitive Predictive Instrument (ACI-TIPI);
includes, but not limited to the analysis of biosensors such as ECG, EEG, breathing, pulse oxyimetry, blood pressure, sound, and motion sensors.

14. The system of claim 1, wherein the means for a node operating within a distributed collaborative processing network include:
computing device;
network connection; and
distributed and collaborative scheduler process.

15. The system of claim 1, wherein the means for a network of wireless and/or wired communication to other processors include:
computing device;
wireless and/or wired communication means;
communications process;
link management for minimizing power consumption, RF radiated power, and error recovery.

16. The system of claim 1, wherein the means for update-able software includes:
computing device;
process for scheduling said update-able software; and
process for installing and running said update-able software.

17. The system of claim 1, wherein the means for water and body fluid resistant casing includes:
computing device casing with means for sealing.

18. The system of claim 1, wherein the means for non-protruding small form factor computing device casing whose external shape and dimensions are non-intrusive.

19. The system of claim 1, wherein the means for rapid and zero insertion force connector includes:
computing device;
connector for the electrical and mechanical joining to a biosensor;
connector for the electrical and mechanical joining to a biosensor harness;
connector whose mechanical means provide for very low connection force.

20. The system of claim 1, wherein the means for mechanical interface to a biosensor harness includes:
a latch whose mechanical means provide for low connection force.

21. The system of claim 1, wherein the means for long term operation power source includes:
battery;
on-body generator;
recharging station.

22. The system of claim 21, wherein the means for on-body generator includes:
a battery;
on-body generator comprised of body movement generator means.

23. A method for the capture, processing, analytics, and communication of biosensor data, comprising the steps of;
operating a body worn computing device;
interfacing to said biosensors;
processing of said biosensor signals;
real-time analysis of biosensor signals;
operating as a node in a distributed collaborative processing network;
wireless and/or wired communicating to other processors in the network;
updating software;
utilizing a water and body fluid resistant casing;
utilizing a non-protruding small form factor casing;
rapid and zero insertion force connecting;
interfacing to a biosensor harness mechanically; and
long term operating power source.

24. The method of claim 23, wherein the steps of a body worn computing device comprising:
affixing said device to a body worn harness;
affixing said device on a arm;
affixing said device on a leg; and
affixing said device on a head.

25. The method of claim 23, wherein the steps of a body worn computing device performing:
bio-signal capture, analyzing, storing, and communicating.

26. The method of claim 23, and wherein the steps of providing a lead transformation process, but not limited to:
processing a 5 lead to 12 standard lead;
processing a EASI to 12 standard lead;
processing alternative disease optimum lead arrays to 12 standard lead.

27. The method of claim 23, wherein the steps of providing a real-time analyzer, but not limited to, an Arrhythmia event classifier.

28. The method of claim 23, wherein the steps of providing a real-time analyzer, but not limited to, an Ischemia event classifier.

29. The method of claim 23, wherein the steps of providing a real-time analyzer, but not limited to, a Myocardial Infarction event classifier.

30. The method of claim 28, further comprising he step of an Acute Cardiac Infarction Time Insensitive Predictive Instrument (ACI-TIPI), a continuous event classifier.

31. The method of claim 23, wherein the steps of providing a body worn computing device comprises:
reporting of health and system events;
alerting locally the patient; and
alerting remotely the physician.

32. The method of claim 23, wherein the steps of providing a body worn computing device includes, but is not limited to:
local data storing;
data forwarding and storing on handhelds;
data forwarding and storing on servers.

33. The method of claim 23, wherein the steps of interfacing to biosensors includes, but not limited to:
adding biosensors such as ECG, EEG, breathing, pulse oxyimetry, blood pressure, sound, and motion sensors.

34. The method of claim 23, wherein the steps of processing said biosensor signals and other system issues including:
health and disease analysis;
system analysis;
bio-signal capturing;
data storing;
alert generation;
user interface displaying; and
communicating.

35. The method of claim 23, wherein the steps of processing in real-time analysis of biosensor signals including:
Ischemia event classifying;
Arrhythmia event classifying;
Myocardial Infarction event classifying;
continuous event classifying such as Acute Cardiac Infarction Time Insensitive Predictive Instrument (ACI-TIPI);
analyzing of biosensors such as ECG, EEG, breathing, pulse oxyimetry, blood pressure, sound, and motion sensors.

36. The method of claim 23, wherein the steps of operating a node within a distributed collaborative processing network comprising:
computing processor;
networking; and
scheduling distributed and collaborative processing.

37. The method of claim 23, wherein the steps of a network of wireless and/or wired communicating to other processors include:
communicating by wireless and/or wired means;
managing links for minimizing power consumption, RF radiated power, and error recovery.

38. The method of claim 23, wherein the steps for updating software comprise:
scheduling said update-able software; and
installing and running said update-able software.

39. The method of claim 23, wherein the steps for waterproofing from bodily fluids comprises:
sealing a computing device case.

40. The method of claim 23, wherein the steps for achieving a non-protruding small form factor computing device casing is to size the external shape and dimensions such that they are non-intrusive.

41. The method of claim 23, wherein the steps for providing a rapid and zero insertion force connector include:
configuring a connector whose mechanical means provide for very low connection force for electrical and mechanical joining from the device to a biosensor and the electrical and mechanical joining to a biosensor harness.

42. The method of claim 23, wherein the steps for providing a mechanical interface for a biosensor harnesses include a latch whose mechanical means provide for low connection force.

43. The method of claim 23, wherein the steps for proving a long-term operation power source includes:
providing a battery;
providing an on-body generator;
providing a recharging station.

44. The method of claim 43, wherein the steps for providing an on-body motion generator comprise:
providing a battery;
providing a generator comprised of body motion generator means;
providing a charging means of the battery from the on-body generator.
